(19) United States
(12) Patent Application Publication
(10) Pub. No.: US 2014/0356853 A1
(43) Pub. Date: Dec. 4, 2014

(54) AUTOMATED NUCLEIC ACID ANALYSIS SYSTEM

(71) Applicant: Samsung Electronics Co., Ltd., Suwon-si (KR)

(72) Inventors: Won-seok CHUNG, Suwon-si (KR); Kak Namkoong, Seoul (KR); Kyung-ho Kim, Seoul (KR); Chin-sung Park, Yongin-si (KR); Joon-ho Kim, Seongnam-si (KR); Joon-sub Shim, Yongin-si (KR); Won-jong Jung, Seongnam-si (KR)

(73) Assignee: Samsung Electronics Co., Ltd., Suwon-si (KR)

(21) Appl. No.: 14/167,583

(22) Filed: Jan. 29, 2014

(30) Foreign Application Priority Data
May 31, 2013 (KR) 10-2013-0063112

(51) Int. Cl. C12Q 3/00

(52) U.S. Cl. CPC C12Q 3/00
USPC 435/3; 435/286.5

(57) ABSTRACT

A nucleic acid analysis system comprising a seating area configured to receive a microfluidic cartridge; a pneumatic module configured to supply a pneumatic pressure or a vacuum to the cartridge when mounted on the seating area; a thermal module configured to control temperature in a predetermined portion of the cartridge when mounted on the seating area; an optic module positioned to irradiate light onto the cartridge when mounted on the seating area, and detect light generated or reflected from a sample inside the cartridge when mounted on the seating area; a fluid sensing module that determines whether a fluid in a predetermined portion of a cartridge mounted on the seating area is in a gaseous state or a liquid state; a scanning module that moves the optic module and the fluid sensing module relative to the seating area; and a control module that controls operations of the pneumatic module, the thermal module, the optic module, the fluid sensing module, and the scanning module, and processes and analyzes data received therefrom.

### CROSS-REFERENCE TO RELATED APPLICATIONS

This application claims the benefit of Korean Patent Application No. 10-2013-0063112, filed on May 31, 2013, in the Korean Intellectual Property Office, the entire disclosure of which is hereby incorporated by reference.

### BACKGROUND

1. Field

The present disclosure relates to nucleic acid analysis systems, and more particularly, to automated nucleic acid analysis systems that may quickly and conveniently test a sample.

2. Description of the Related Art

With the advent of the point-of-care age, gene analysis, in vitro diagnosis, and gene base sequence analysis have become increasingly significant, and demand therefor is increasing gradually. In particular, since a nucleic acid-based molecular diagnosis has excellent accuracy and sensitivity, applications thereof in pharmacogenomics and diagnosis of cancers and infectious diseases have increased.

In general, the nucleic acid-based molecular diagnosis is performed using microfluidic devices such as Lab-on-a-Chip devices. A microfluidic device including a plurality of microchannels and microchambers is designed to control and operate microfluids (for example, several nl to several ml). By using a microfluidic device, the reaction time of microfluids may be minimized, and the reaction of microfluids and the measurement of results thereof may be performed in a single microfluidic device.

For example, nucleic acid analysis using a microfluidic device may include: flowing a sample to a predetermined position inside the microfluidic device so that a target cell may be captured, washing off impurities captured together with the target cell; disrupting the captured cell; moving a solution mixed with the disrupted cell to a predetermined position inside the microfluidic device; mixing the solution mixed with the disrupted cell with components necessary for amplification of a nucleic acid; amplifying an extracted nucleic acid; and detecting the amplified nucleic acid. There remains a need for analysis systems for automatically performing these operations.

### SUMMARY

According to an aspect of an exemplary embodiment of the present invention, a nucleic acid analysis system includes: a pneumatic module that generates a pneumatic pressure or a vacuum and supplies the pneumatic pressure or the vacuum to a cartridge including a microfluidic device; a thermal module that controls a temperature of a predetermined portion inside the cartridge; an optic module that irradiates light onto the cartridge and detects light generated from a sample inside the cartridge; a fluid sensing module that determines whether a fluid existing at the predetermined portion inside the cartridge is in a gaseous state or a liquid state; a scanning module that moves the optic module and the fluid sensing module; and a control module that controls operations of the pneumatic module, the thermal module, the optic module, the fluid sensing module, and the scanning module and processes and analyzes data obtained therefrom.

For example, the fluid sensing module may include: a light source that emits light toward the cartridge and a photodetector that detects light reflected from the cartridge. The light source may include a light emitting diode or a laser diode, and the photodetector may include a photodiode, a photomultiplier tube, a phototransistor, a charge-coupled device (CCD) image sensor, or a complementary metal-oxide-semiconductor (CMOS) image sensor. The fluid sensing module may further include a reflector that reflects light that passed through the cartridge to the photodetector. The light source and the photodetector may be disposed in the same direction with respect to the cartridge, and the reflector may be disposed on an opposite side of the light source and the photodetector with respect to the cartridge.

In an embodiment, the control module may be configured to determine a state of a fluid by comparing a signal obtained from reflected light measured by the fluid sensing module with a reference value. The control module may be configured to determine a state of a fluid by comparing a reference value with an average value of a plurality of data about a signal obtained from reflected light measured just previously by the fluid sensing module, including current data about a signal obtained from reflected light measured by the fluid sensing module. The control module may also be configured to determine a state of a fluid by comparing a reference value with a difference between an average value of a plurality of data about a signal obtained from reflected light measured most recently by the fluid sensing module and an average value of a plurality of data about a signal obtained from reflected light measured a predetermined number of times previously by the fluid sensing module. The control module may also be configured to determine a state of a fluid by comparing a reference value with a differential value calculated using a signal obtained from reflected light measured most recently by the fluid sensing module and a signal obtained from reflected light measured a predetermined number of times previously by the fluid sensing module. The control module may also be configured to determine a state of a fluid by comparing a reference value with a differential value calculated using an average value of a plurality of data obtained from reflected light measured most recently by the fluid sensing module and an average value of a plurality of data obtained from reflected light measured a predetermined number of times previously by the fluid sensing module.

In an embodiment, the control module may be configured to determine that a state of a fluid has changed after a signal indicating that a state of a fluid has changed occurs consecutively at least a predetermined number of measurement times.

The pneumatic module may include: a pneumatic pump that generates a pneumatic pressure; a vacuum pump that generates a vacuum; a chamber that is kept under the pneumatic pressure and the vacuum; a regulator that regulates the pressure of the chamber; a pressure sensor that measures the pneumatic pressure and the vacuum of the chamber; a plurality of pneumatic ports that are configured to inject a pneumatic pressure and a vacuum into the cartridge; and a plurality of valves that are configured to provide a pneumatic pressure or a vacuum to a selected pneumatic port.

The thermal module may include: a heating unit that is configured to raise a temperature of the cartridge; a cooling unit that is configured to reduce a temperature of the cartridge; and a temperature sensor that is configured to measure a temperature. For example, the heating unit may include a resistive heater or a Peltier element; the cooling unit may include a cooling fan, a blower, or a Peltier element; and the temperature sensor may include a resistance temperature detector (RTD), a thermistor, a thermocouple, or an infrared (IR) sensor.

The optic module may, for example, detect amplified nucleic acids in the cartridge by fluorescence detection. In an embodiment, the optic module may include: a light source that generates excitation light and irradiates the excitation light onto the cartridge; and a photodetector that detects fluorescence generated from a fluorescent dye marked on the sample.

The scanning module may include, for example: a motor and a lead screw rotated by the motor. The optic module and the fluid sensing module may be coupled to the scanning module.

In another embodiment, the scanning module may include: a motor and a pulley and belt connected to the motor. Alternatively, the scanning module may include a linear motor including: a magnet, a voice coil, and an encoder.

The control module may include: a microprocessor; an algorithm that is configured to control the pneumatic module, the thermal module, the optic module, the fluid sensing module, and the scanning module and analyze data; and control software programmed with a user interface.

According to an aspect of the present invention, a nucleic acid analysis method using the above nucleic acid analysis system includes: disrupting a sample to be analyzed; purifying the sample to be analyzed; forming a mixed solution by mixing the disrupted and purified sample with materials necessary for nucleic acid amplification; and amplifying and detecting a nucleic acid by controlling a temperature of the mixed solution. The sample may include, for example, a swab, a culture medium, saliva, sputum, a cellular tissue, urine, stool, blood, pus, or cerebrospinal fluid. The disrupting of the sample may include, for example, mechanical disruption, chemical disruption, thermal disruption, or a combination thereof. The controlling of the temperature of the mixed solution may include any one or a combination of: maintaining a constant temperature for a predetermined time; changing a temperature within a predetermined range for a predetermined time; and repeatedly maintaining a plurality of different temperatures for respective predetermined times.

### BRIEF DESCRIPTION OF THE DRAWINGS

These and/or other aspects will become apparent and more readily appreciated from the following description of the embodiments taken in conjunction with the accompanying drawings, in which:

FIG. 1 is a block diagram illustrating a schematic configuration of a nucleic acid analysis system;

FIGS. 2A and 2B are schematic perspective views illustrating an exemplary configuration of the nucleic acid analysis system illustrated in FIG. 1;

FIG. 3 is a perspective view illustrating a scanning module of the nucleic acid analysis system illustrated in FIGS. 1 and 2, and an optic module and a fluid sensing module coupled to the scanning module;

FIG. 4 is a plan view illustrating a structure of a microfluidic cartridge including a plurality of microchannels and reaction chambers;

FIG. 5 is a conceptual diagram illustrating a principle of a fluid state sensing operation;

FIG. 6 is a graph illustrating an optical signal obtained by scanning a microfluidic device;

FIG. 7 is a graph illustrating a change in the optical signal according to a change in the fluid state inside the microchannel;

FIG. 8 is a graph illustrating a variation in the optical signal when bubbles exist in a liquid flowing through the microchannel;

FIGS. 9A and 9B are graphs illustrating the results of processing the optical signal according to an algorithm for determining that the fluid state inside the microchannel has changed;

FIG. 10 is a graph illustrating an algorithm for suppressing a determination error caused by bubbles inside a liquid or droplets inside a gas;

FIG. 11 is a perspective view illustrating a thermal module of the nucleic acid analysis system illustrated in FIG. 1; and

FIG. 12 is a graph illustrating results of analyzing a nucleic acid by the nucleic acid analysis system illustrated in FIG. 1.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Hereinafter, automated nucleic acid analysis systems according to exemplary embodiments will be described with reference to the accompanying drawings. The sizes of respective elements in the drawings may be exaggerated for the sake of clarity and convenience.

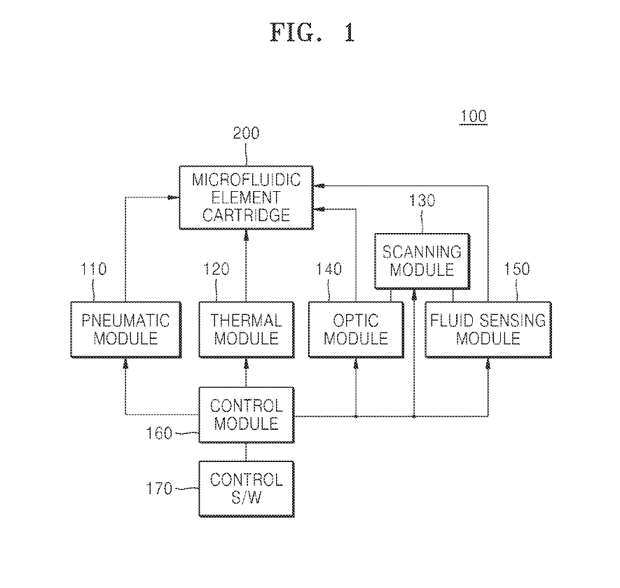

FIG. 1 is a block diagram illustrating a schematic configuration of a nucleic acid analysis system according to an embodiment. Referring to FIG. 1, a nucleic acid analysis system 100 according to an embodiment may automatically perform a series of operations of processing various samples in a microfluidic cartridge 200 (hereinafter referred to as “the cartridge 200”) of a microfluidic device, including extracting a nucleic acid from the sample, amplifying the extracted nucleic acid, and analyzing the amplified nucleic acid. To this end, the nucleic acid analysis system 100 may include a pneumatic module 110, a thermal module 120, a scanning module 130, an optic module 140, a fluid sensing module 150, and a control module 160. The cartridge 200 may be detachably disposed within the nucleic acid analysis system 100. Thus, when completely analyzed, the cartridge 200 may be removed and replaced with another cartridge 200. Although not illustrated in FIG. 1, the nucleic acid analysis system 100 may further include a deck for mounting/dismounting the cartridge 200, and a pneumatic port of the pneumatic module 110, the thermal module 120, and the scanning module 130 may be disposed around the deck.

The pneumatic module 110 generates a pneumatic pressure and a vacuum, controls the pneumatic pressure and the vacuum, and supplies the controlled pneumatic pressure and vacuum to the cartridge 200. For example, the pneumatic module 110 may include: a pneumatic pump that generates a pneumatic pressure; a vacuum pump that generates a vacuum; a regulator that regulates a pressure to a suitable value; a pressure sensor that measures the pneumatic pressure and vacuum; a chamber that is kept under the pneumatic pressure and vacuum and is controlled to maintain a pressure within a predetermined range; a plurality of pneumatic ports that are configured to inject a pneumatic pressure and a vacuum into the cartridge 200; and a plurality of valves that are configured to provide a pneumatic pressure or a vacuum to a selected pneumatic port. The above components of the pneumatic module 110 are not necessarily disposed together at a predetermined position inside the nucleic acid analysis system 100, and some components may be distributively disposed at a plurality of positions inside the nucleic acid analysis system 100 according to requirements for design and function.

The thermal module 120 controls a temperature of a predetermined portion inside the cartridge 200. To this end, the thermal module 120 may include: a heating unit that is configured to raise a temperature of the cartridge 200; a cooling unit that is configured to reduce a temperature of the cartridge 200; and a temperature sensor that is configured to measure a temperature. Examples of the heating unit may include a resistive heater and a Peltier element. Examples of the cooling unit may include a cooling fan, a blower, and a Peltier element. Examples of the temperature sensor may include a resistance temperature detector (RTD), a thermistor, a thermocouple, and an infrared (IR) sensor.

The optic module 140 irradiates light into the cartridge 200, and detects light generated from the sample inside the cartridge 200. For example, according to a fluorescence detection method, the optic module 140 may irradiate an excitation light of a predetermined wavelength onto the sample inside the cartridge 200 and detect fluorescence generated from a fluorescent dye labeled on the sample. The optic module 140 may include a light source, a photodetector, an optical lens, and an optical filter. Examples of the light source may include a light emitting diode (LED) and a laser diode (LD), and examples of the photodetector may include a photodiode (PD), a photomultiplier tube (PMT), a phototransistor, a charge-coupled device (CCD) image sensor, and a complementary metal-oxide-semiconductor (CMOS) image sensor.

The fluid sensing module 150 senses a movement of a fluid inside the cartridge 200. The fluid sensing module 150 determines whether a fluid existing at a predetermined portion inside the cartridge 200 is in a gaseous state or a liquid state.

The fluid sensing module 150 may determine whether a fluid existing at a predetermined portion inside the cartridge 200 is in a gaseous state or a liquid state by irradiating light or an electromagnetic wave onto a predetermined portion of the cartridge 200 and sensing a change in the intensity of light or electromagnetic wave reflected from the cartridge 200. According to this embodiment, by using the fluid sensing module 150, the state of a fluid existing in a region of the cartridge 200 may be determined quickly and accurately. Therefore, since the state of a fluid at various positions of the cartridge 200 may be quickly determined, the control module 160 of the nucleic acid analysis system 100 may control the flow of the fluid accurately according to circumstances. The configuration and operation of the fluid sensing module 150 will be described later in more detail.

The scanning module 130 moves the optic module 140 and the fluid sensing module 150 to a desired position. For example, under the control of the control module 160, the scanning module 130 may move the optic module 140 and the fluid sensing module 150 to a desired position on the cartridge 200. The scanning module 130 may include, for example, a motor, a lead screw, a transfer guide, and a position sensor.

In order to analyze the sample inside the cartridge 200, the control module 160 controls operations of modules 110, 120, 130, 140, and 150. Also, the control module 160 may process, analyze, and feed back data obtained from the operations of modules 110, 120, 130, 140, and 150, and may calculate and output analysis data. To this end, the control module 160 may include: a microprocessor; an algorithm for controlling modules 110, 120, 130, 140, and 150 and analyzing data; and control software programmed with a user interface.

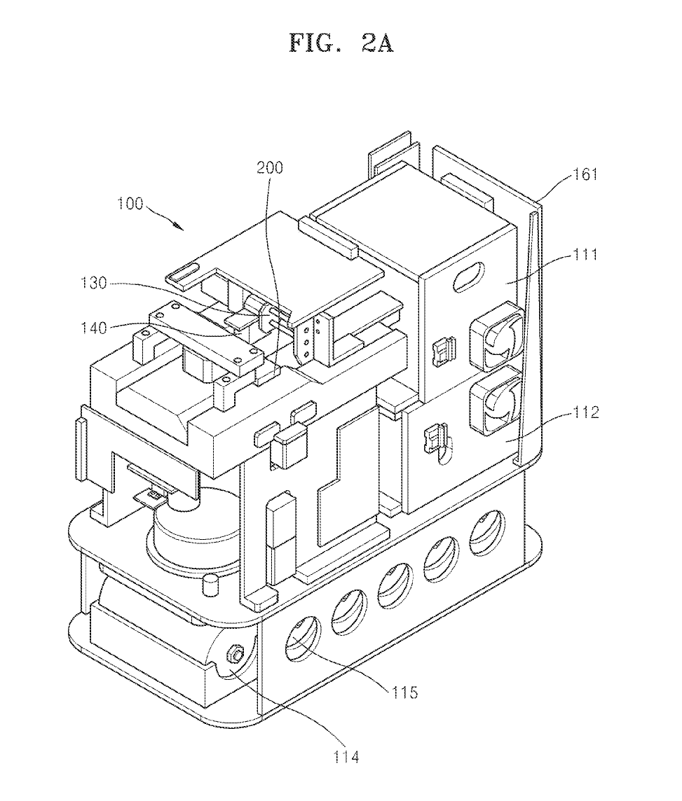

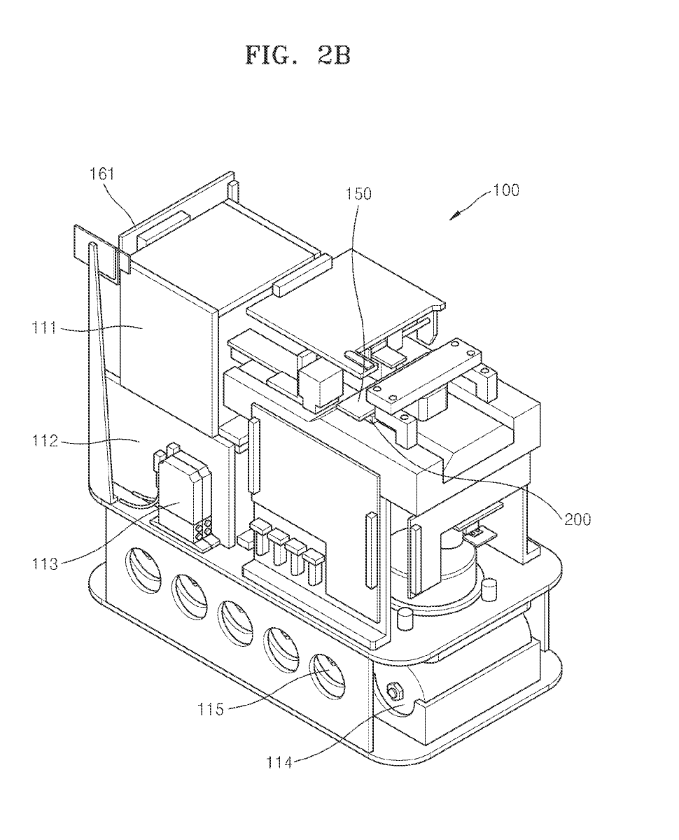

FIGS. 2A and 2B are schematic perspective views illustrating an exemplary configuration of the nucleic acid analysis system 100 illustrated in FIG. 1. FIG. 2A mainly illustrates a right side of the nucleic acid analysis system 100, and FIG. 2B mainly illustrates a left side of the nucleic acid analysis system 100. The configuration of the nucleic acid analysis system 100 illustrated in FIGS. 2A and 2B is an example, and this embodiment is not limited to the configuration illustrated in FIGS. 2A and 2B.

Referring to FIGS. 2A and 2B, a pneumatic pressure and a vacuum generated by a pneumatic pump 111 and a vacuum pump 112 may be injected through input ports of chambers 114 and 115, respectively. Thus, the pneumatic pump 111 and vacuum pump 112, together part of the pneumatic module, may be connected to the chambers (e.g., fluidly connected; connections not shown). The internal pressures of the chambers 114 and 115 may be monitored by pressure sensors connected to the chambers 114 and 115, respectively. When the internal pressures of the chambers 114 and 115 deviate from a predetermined range, the control module 160 may operate the pneumatic pump 111 or the vacuum pump 112 to maintain the internal pressures of the chambers 114 and 115 within the predetermined range.

A regulator 113 is connected to the chambers 114 and 115. The control module 160 may control the regulator 113 to maintain the pneumatic pressure and vacuum in the cartridge 200 at desired levels. Thus, pneumatic pump 111 and/or vacuum pump 112 may be connected (e.g., fluidly connected) to the seating area (not shown in FIGS. 2A and 2B because the seating area is covered by the cartridge 200) of the cartridge 200 and, thus, the cartridge 200 itself when positioned on the seating area, by way of the chambers 114 and 115. For example, a plurality of solenoid valves (not illustrated) may be used to supply a pneumatic pressure and a vacuum only to a desired portion of the cartridge 200. The pneumatic pressure and the vacuum supplied to the cartridge 200 may be used to move and mix a fluid inside the cartridge 200 and to extract a nucleic acid inside the sample. The control module 160 may sense a movement of the fluid inside the cartridge by the fluid sensing module 150 and move the fluid to a desired position in a predetermined sequence.

Since the optic module 140 and the fluid sensing module 150 are mounted on the scanning module 130, they may be moved to a desired position under the control of the control module 160. The control module 160 may be implemented, for example, as software stored on a control board 161 that includes microprocessors.

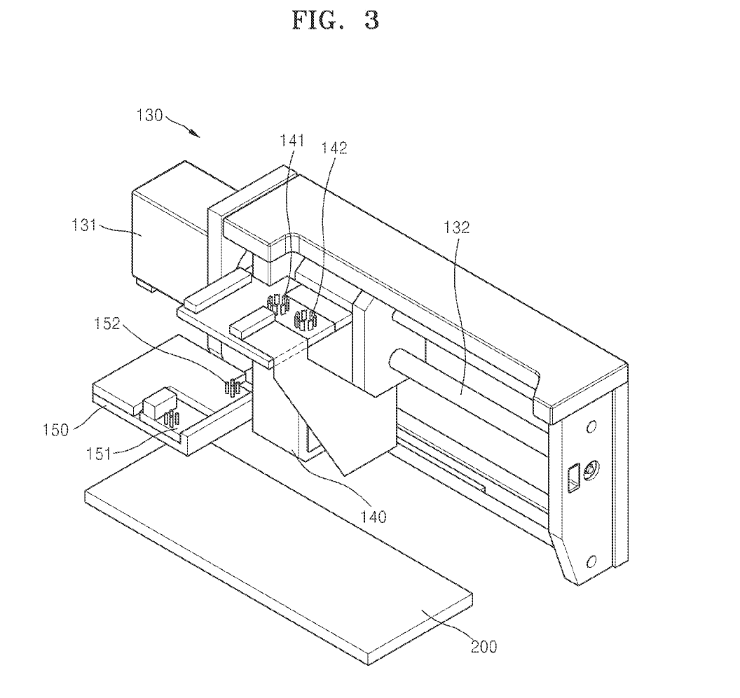

FIG. 3 is a perspective view illustrating examples of the scanning module 130 of the nucleic acid analysis system 100 illustrated in FIGS. 1 and 2, and the optic module 140 and the fluid sensing module 150 coupled to the scanning module 130. Referring to FIG. 3, the scanning module 130 may include: a step motor 131; and a lead screw 132 rotated by the step motor 131. Since both the optic module 140 and the fluid sensing module 150 are coupled to the lead screw 132, they may make a horizontal linear movement according to a rotary motion of the lead screw 132. Therefore, by rotating the lead screw 132 using step motor 131, the optic module 140 and the fluid sensing module 150 may be accurately moved to a desired position. As illustrated in FIG. 3, the optic module 140 may include a light source 141 and a photodetector 142 for fluorescence detection, and the fluid sensing module 150 may also include a light source 151 and a photodetector 152. Since the cartridge 200 may be disposed under the optic module 140 and the fluid sensing module 150, the optic module 140 and the fluid sensing module 150 may irradiate light to a predetermined position of the cartridge 200.

FIG. 3 illustrates that the scanning module 130 includes the step motor 131 and the lead screw 132; however, these features are exemplary and the present embodiment is not limited thereto. For example, the scanning module 130 may include a pulley and a belt connected to the step motor 131, instead of the lead screw 132. Alternatively, the scanning module 130 may include a linear motor including a magnet, a voice coil, and an encoder.

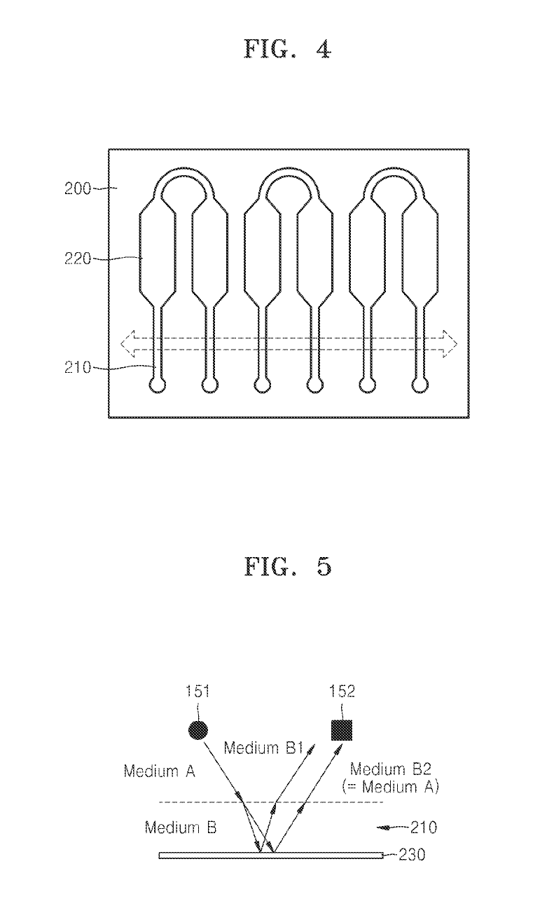

FIG. 4 is a plan view illustrating a structure of a microfluidic cartridge 200 including a plurality of microchannels 210 and reaction chambers 220. As illustrated in FIG. 4, the cartridge 200 may include a plurality of microchannels 210 through which a fluid such as a sample or a reagent flows, and a plurality of reaction chambers 220 in which a reaction of the sample and the reagent occurs. Although not illustrated in FIG. 4 in detail, in addition to the microchannels 210 and the reaction chambers 220, the cartridge 200 may further include a plurality of microvalves for controlling the flow of a fluid inside the microchannels 210, a plurality of pneumatic chambers that are connected to the respective microchannels 210 and the respective microvalves, and a plurality of openings for injecting and discharging a fluid and a pneumatic pressure inside the cartridge 200. However, for the convenience of description, only the microchannels 210 and the reaction chambers 220 are illustrated in FIG. 4.

Under the control of the control module 160, the pneumatic module 110 may apply a vacuum or a pneumatic pressure to the microchannels 210 or the microvalves through the openings of the cartridge 200 to pull or push a fluid inside the microchannels 210 or to open/close the microvalves. The control module 160 may determine the fluid state inside each of the microchannels 210 through the fluid sensing module 150 and control the pneumatic module 110 based on a determination result of the fluid sensing module 150 to move a fluid inside the cartridge 200 to a desired position. To this end, the control module 160 may determine whether a fluid or a gas flows through the microchannels 210 by scanning the microchannels 210 while moving the fluid sensing module 150 to a desired position using the scanning module 130.

A fluid state sensing operation of the nucleic acid analysis system 100 according to this embodiment will be described below in more detail. FIG. 5 is a conceptual diagram illustrating a principle of the fluid state sensing operation. Referring to FIG. 5, the light source 151 and the photodetector 152 are disposed over the microchannel 210, and a reflector 230 is disposed under the microchannel 210. The reflector 230 reflects light that passes through the microchannel 210 of the cartridge 200 to the photodetector 152 such that a sufficient amount of light is input to the photodetector 152. For example, it is assumed that a first medium A such as air exists between the light source 151 and the photodetector 152 and the microchannel 210, and a second medium B flows through the microchannel 210.

When the second medium B flowing though the microchannel 210 of the cartridge 200 is air like the first medium A, light emitted from the light source 151 is not refracted at an interface between the first medium A and the second medium B. However, when the second medium B changes into a liquid having a different refractive index than the first medium A, since the light is refracted at an interface between the first medium A and the second medium B, a travel path of the light also changes. As a result, since the amount of light traveling toward the photodetector 152 varies, it may be detected from a change in the light amount detected by the photodetector 152 that a material flowing through the microchannel 210 of the cartridge 200 changes, for example, from gas to liquid or from liquid to gas.

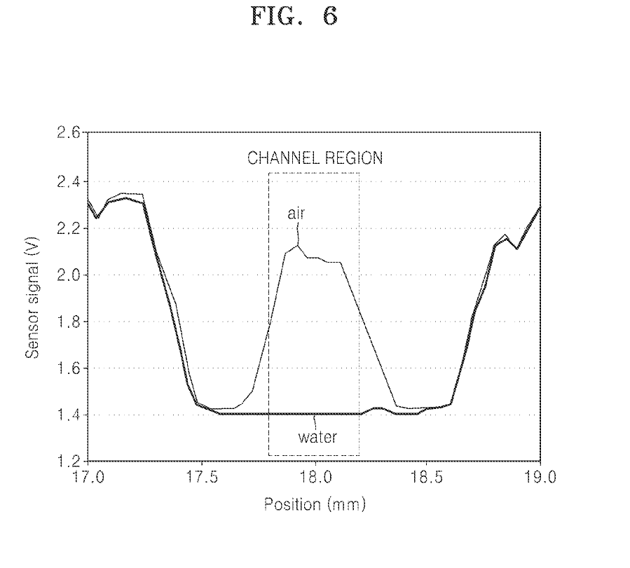

FIG. 6 is a graph illustrating an optical signal obtained by scanning the cartridge 200 using fluid sensing module 150. The graph of FIG. 6 was obtained by scanning any one of the microchannels 210 of the cartridge 200 from the left side to the right side.

The cartridge 200 was formed of polystyrene (PS), the depth and width of the microchannel 210 were respectively about 300 μm and about 400 μm, and a laser having a center wavelength of about 850 nm was used as the light source 151. Also, as the reflector 230, a translucent silicon material was brought into close contact with the bottom surface of the microchannel 210. In FIG. 6, the region indicated by a dotted-line box represents a region of the microchannel 210. Referring to FIG. 6, it may be seen that an optical signal decreases when water flows through the microchannel 210 as compared with when air flows therethrough. Therefore, whether a fluid inside the microchannel 210 is water or air may be determined by selectively processing only data of coordinates corresponding to the region of the microchannel 210. In this manner, by scanning the microfluid channels 210 one by one, parallel fluid control of the microchannels 210 may be performed.

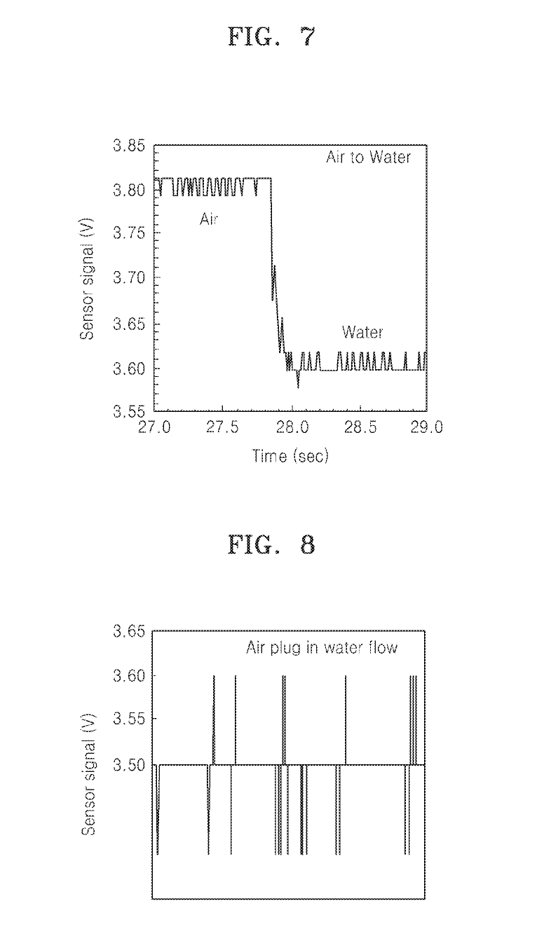

FIG. 7 is a graph illustrating a change in the optical signal according to a change in the fluid state inside the microchannel 210. The graph of FIG. 7 illustrates the measurement of a change in an optical signal when a fluid inside the microchannel 210 changes from air to water after the light source 151 and the photodetector 152 are fixed to the selected microchannel 210. Herein, the depth and width of the microchannel 210 were respectively about 100 μm and about 400 μm, and the sampling rate was about 100 Hz. Referring to FIG. 7, as a fluid inside the microchannel 210 changes from air to water, the optical signal becomes smaller in amplitude. Therefore, while monitoring the fluid state at one point of the cartridge 200, the nucleic acid analysis system 100 may sense the moment when the fluid changes from air to water or from water to air and may suitably control the fluid inside the cartridge 200 based on the sensed information. For example, when the strength of an optical signal output from the photodetector 152 is equal to or higher than a predetermined reference value, the control module 160 may determine that the fluid is gas. When the strength of the optical signal is less than the predetermined reference value, the control module 160 may determine that the fluid is liquid.

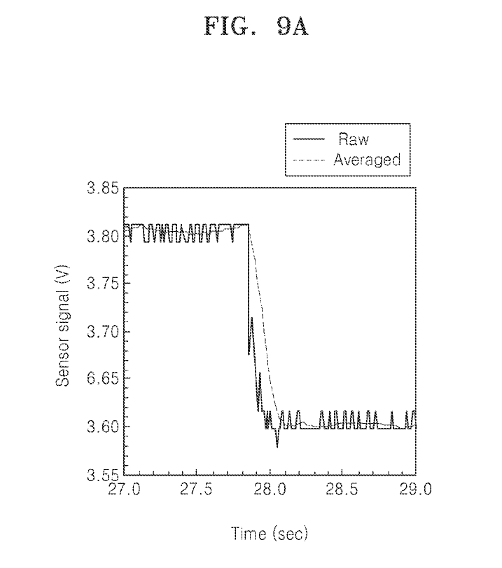

However, as illustrated in FIG. 7, a non-uniform noise component may occur in the optical signal even while the fluid maintains a steady state. In general, this noise component is caused by droplets mixed in gas or bubbles included in liquid. FIG. 8 illustrates a variation in the optical signal when bubbles are included in a liquid (e.g., water) flowing through the microchannel 210. Due to this noise component, it may be difficult to accurately determine the time point when the fluid state changes. Also, the optical signal difference between the fluid being water and the fluid being air may change according to various factors such as the size of a measurement point (e.g., the microchannel 210), the distance from the fluid sensing module 150 to the measurement point, and the state of the reflector 230. Therefore, when bubbles are mixed in water flowing through the measurement point or droplets are included in air flowing through the measurement point, an incorrect determination may be made before the fluid state changes completely.

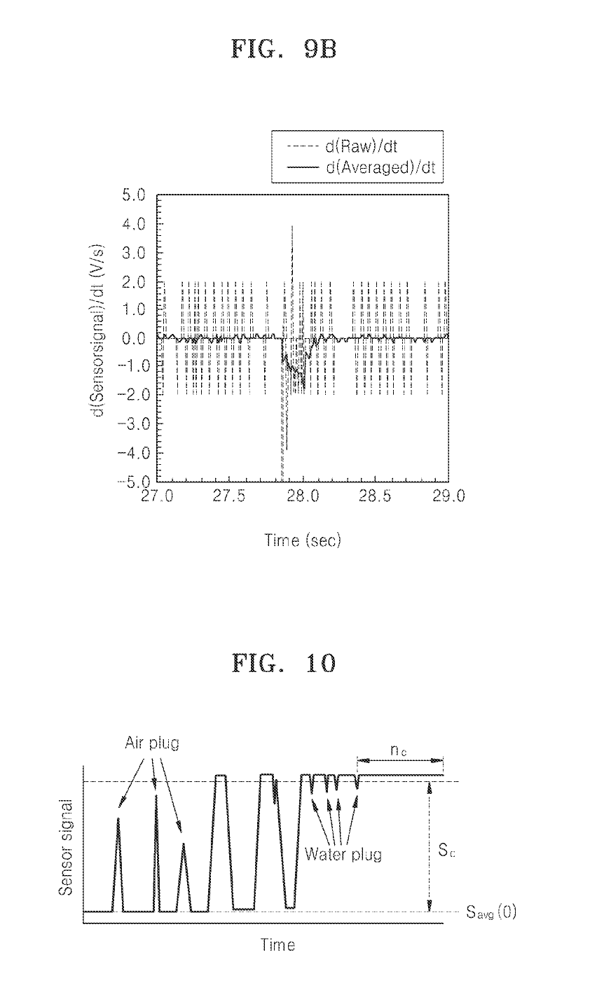

Hereinafter, an algorithm is presented for accurately determining the time point when a fluid change occurs, even in the above-described situation. First, a method of averaging a plurality of data may be used as a method for reducing the influence of a noise component. For example, in FIG. 9A, the thin dotted line represents an average of sixteen previous data with respect to one point of a graph of raw data (represented by the thick solid line). As illustrated in FIG. 9A, the noise component is reduced when the data averaging method is used. The fluid state may be determined by comparing an averaged value (as shown by the thin dotted line) with a reference value. For example, the data averaging method may be expressed as Equation 1 below.

$${S}_{c} \leq {S}_{avg} ( n ) = \frac{{\sum }_{k = 1}^{{n}_{a}} S ( [ n - ( k - 1 ) ] )}{{n}_{a}} \qquad [ Equation 1 ]$$

That is, as expressed in Equation 1, it may be possible to determine whether a fluid currently included in the microchannel 210 is gas (e.g., air) or liquid (e.g., water, sample, or reagent), by comparing an average value S_{avg}(n) of nₐ data previously measured, including current data, with a predetermined reference value S_{c}. Herein, the reference value S_{c} is may be a predetermined fixed value, or may be determined in each measurement based on data measured before the fluid state changes. For example, in each measurement, the average value S_{avg}(n) immediately after the fluid changes from liquid to gas and the average value S_{avg}(n) immediately after the fluid changes from gas to liquid may be used as the reference value S_{c}.

A variation of the average value may be used instead of the average value. For example, when the difference between the average value of optical signals most recently obtained and the average value of optical signals previously obtained is greater than a reference variation, the control module 160 may determine that the fluid state has changed. This method may be expressed as Equation 2 below.

ΔS_{c}≦|S_{avg}(n)−S_{avg}(n₀)| [Equation 2]

In Equation 2, S_{avg}(n) is an average value of nₐ data most recently measured, and S_{avg}(n₀) is an average value of nₐ data measured a predetermined number of times before S_{avg}(n). That is, when the absolute value of the difference between the average value of data most recently measured and the average value of data measured a predetermined number of times previously is equal to or greater than a predetermined reference variation ΔS_{c}, the control module 160 may determine that the fluid state has changed.

As another method, a determination of fluid state may be made based on temporal differential values of data. For example, in FIG. 9B, the dotted line represents the temporal differentiation of the raw data illustrated in FIG. 9A, and the solid line represents the temporal differentiation of the averaged value plot illustrated in FIG. 9A. As shown by the dotted line, the differential value varies greatly with time, and in particular, the variation value increases further at the moment when the fluid state changes from gas to liquid. As shown by the solid line, the differential value varies little while the fluid state does not change, and varies significantly only while the fluid state changes from gas to liquid. Therefore, the control module 160 determines that the fluid state has changed by comparing a variation of the differential value with a predetermined reference variation. In particular, when the averaged data are differentiated, a fluid state change may be determined more easily. This differential equation may be expressed, for example as Equation 3 below.

$$Δ {S}_{c}^{′} \leq \frac{\vert {S}_{avg} ( n ) - {S}_{avg} ( {n}_{0} ) \vert}{{t}_{n} - {t}_{0}} \qquad [ Equation 3 ]$$

In Equation 3, tₙ is the time when S_{avg}(n) is obtained, and t₀ is the time when Savg(n₀) is obtained. That is, when a value, obtained by dividing the absolute value of the difference between the average value of data most recently measured and the average value of data measured a predetermined number of times previously by time, is equal to or greater than a predetermined reference variation ΔS′_{c}, the control module 160 may determine that the fluid state has changed.

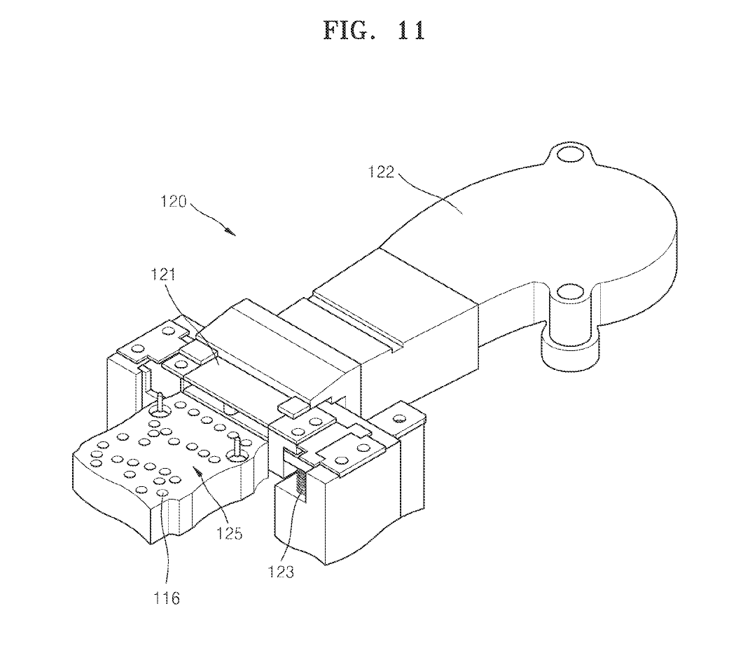

In order to prevent a determination error caused when bubbles exist in liquid or droplets exist in gas, the control module 160 may determine that the fluid state has changed only when the state of a predetermined number or more of data greater than or smaller than a predetermined reference value is maintained continuously. For example, referring to FIG. 10, it is assumed that the fluid inside the microchannel 210 is initially in a liquid state and the fluid changes into a gaseous state after a predetermined time as air flows into the microchannel 210. An average data value of initial optical signals measured before the inflow of air is S_{avg}(0). Thereafter, when air flows into the microchannel 210, the average value of optical signals increases above the reference value Sc. However, since many droplets still exist in the microchannel 210, the data may vary around the reference value. Therefore, when the data vary around the reference value, the control module 160 may determine that fluid state has changed, and when the values of optical signals measured consecutively at least a predetermined number of times n_{c}, or the average value of the optical signals is both equal to or greater than the reference value, the control module 160 may determine that fluid in the microchannel 210 has changed completely from liquid to gas. That is, when a signal indicating a fluid state change occurs at least a predetermined number of measurement times, the control module 160 may finally determine that the fluid state has changed.

The above fluid state determination method may be performed on any one measurement point of the cartridge 200, or may be performed by scanning a plurality of measurement points. When the fluid state is monitored by scanning a plurality of measurement points, an independent determination may be made on each of the measurement points, or a determination may be made by synthesizing the measurement results of the measurement points. For example, after moving the fluid sensing module 150 using the scanning module 130 to any measurement point of the cartridge 200 and monitoring the fluid state, the control module 160 may move the fluid sensing module 150 to the next measurement point. In this case, when detecting a fluid state change at any measurement point, the control module 160 may determine that the fluid state has changed only at the point. Alternatively, after scanning a plurality of measurement points within a section where the fluid flows, when sensing that the fluid state at all of the measurement points has changed, the control module 160 may determine that the fluid state in the section has changed.

When monitoring the fluid state at a plurality of measurement points, after monitoring the respective measurement points for only a predetermined time, the control module 160 may monitor the next measurement point. Upon completion of the monitoring of all the measurement points, the control module 160 monitors the first measurement point again. In this case, the control module 160 may determine the fluid state at the measurement point by comparing the previous measurement result at the measurement point with the current measurement result.

FIG. 11 is a perspective view illustrating a configuration of the thermal module 120 of the nucleic acid analysis system 100 illustrated in FIG. 1. Since the thermal module 120 may be disposed inside the assembled nucleic acid analysis system 100, the thermal module 120 does not appear in the perspective views of FIGS. 2A and 2B. The thermal module 120 may include a heating unit 121 that is disposed under a seating area 125 on which the cartridge 200 (see FIGS. 2A and 2B) is to be seated, and a cooling unit 122 that is disposed to blow cooling air toward the seating area 125. The heating unit 121 raises the temperature of a predetermined portion inside the cartridge 200 to a desired level, and the cooling unit 122 reduces the raised temperature to a desired level. In order to improve the contact between the heating unit 121 and the cartridge 200, the heating unit 121 may be supported by, for example, a pressure member 123. Then, the thermal transmission performance from the heating unit 121 to the cartridge 200 may be improved. Also, as illustrated in FIG. 11, a plurality of pneumatic ports 116 may be disposed under the seating area 125 on which the cartridge 200 is mounted. The pneumatic pump 111 and the vacuum pump 112 of the pneumatic module 110 (see FIGS. 2A and 2B) may supply a pneumatic pressure and a vacuum necessary for a fluid transfer inside the cartridge 200 to the cartridge 200 through the pneumatic ports 116.

A process of analyzing a nucleic acid inside a sample using the nucleic acid analysis system 100 will be described below. First, the cartridge 200 including the sample is installed in the nucleic acid analysis system 100. The sample may be, for example, a swab, a culture medium, saliva, sputum, a cellular tissue, urine, stool, blood, pus, or cerebrospinal fluid. In order to target cells included in the sample, the sample is flowed to a predetermined position inside the cartridge 200. This operation may be performed by selectively supplying a pneumatic pressure and a vacuum to each of the pneumatic ports 116 by the pneumatic module 110 under the control of the control module 160. In this case, the fluid sensing module 150 may be used to determine whether the sample is transferred to a desired position. When target cells are completely captured, the captured target cells are fixed. Thereafter, impurities captured together with the target cells inside the sample may be washed off, and a space in which the target cells are captured may be dried. This purification operation may also be performed by operating the pneumatic module 110 and the fluid sensing module 150 under the control of the control module 160.

The space in which the target cells are captured is then filled with a solution necessary for disrupting the target cell, and the target cell may be disrupted using various methods. The target cell may be disrupted by, for example, mechanical disruption, chemical disruption, thermal disruption, or a combination thereof. The order of the purification operation for removing impurities and the target cell disruption operation may be changed. That is, the impurity purification may be performed after the target cell disruption.

After the target cells are disrupted, a solution mixed with the disrupted target cells may be moved to a predetermined position inside the cartridge 200, and the solution mixed with the disrupted target cells may be mixed with materials necessary for nucleic acid amplification. Thereafter, the nucleic acid inside the disrupted target cell may be amplified by, for example, polymerase chain reaction (PCR). During the PCR operation, the thermal module 120 may be used to control the temperature of a predetermined position (for example, a PCT occurrence position) inside the cartridge 200. The temperature control may be performed in various ways according to the types of samples and the detection methods. For example, during the amplification of the nucleic acid, a process of raising the temperature to a desired level and then reducing the temperature to a desired level may be repeated. An operation of maintaining the temperature for a predetermined time, after the temperature was raised to the desired level, and then reducing the temperature, and maintaining the temperature for a predetermined time after the temperature was reduced to a desired level may be repeated. Instead of controlling only two temperatures, an operation of maintaining a plurality of different temperatures for respective predetermined times may be repeated. Also, the temperature for amplifying the nucleic acid may be maintained at a predetermined level for a predetermined time, or the temperature may be changed within a predetermined range for a predetermined time.

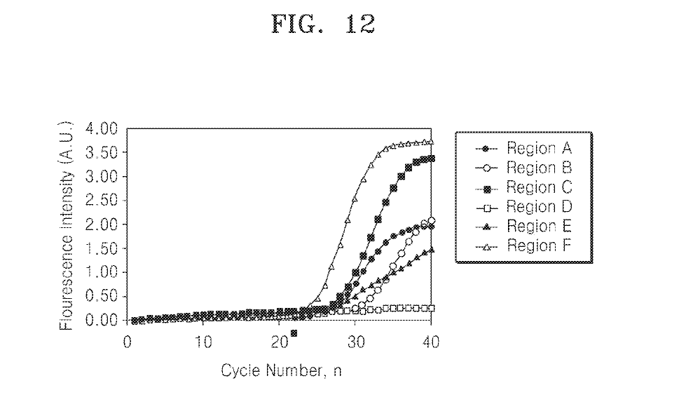

Whenever one PCR cycle of raising and then reducing the temperature at a predetermined position inside the cartridge 200 is completed, the optic module 140 may be used to perform nucleic acid analysis. The optic module 140 may detect the amplified nucleic acids by, for example, fluorescence detection. FIG. 12 is a graph illustrating the results of analyzing a nucleic acid by fluorescence detection using the nucleic acid analysis system illustrated in FIG. 1. In the graph of FIG. 12, the vertical axis represents the intensity of fluorescence measured in a plurality of regions in which nucleic acids are arranged, and the horizontal axis represents a PCR cycle count. As apparent from the graph of FIG. 12, as the PCR cycle count increases, the number of amplified nucleic acids increases, and thus, the intensity of fluorescence may increase gradually. However, in the case of a region D, it may be seen that since there is no nucleic acid in the sample, no nucleic acid is amplified. In the case of the other regions, since nucleic acid exists in the sample, the control module 160 may analyze the nucleic acid inside the sample in consideration of the position in the PCR cycle in which the intensity of fluorescence increases, and the intensity of fluorescence in each region after completion of the amplification.

Exemplary embodiments of the automated nucleic acid analysis system have been described and illustrated in the accompanying drawings. However, it should be understood that the exemplary embodiments should be considered in a descriptive sense only and not for purposes of limitation. It should also be understood that the present invention is not limited to the above description and illustration and various changes may be made therein by those skilled in the art. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A nucleic acid analysis system comprising:
a seating area configured to receive a microfluidic cartridge;
a pneumatic module configured to supply a pneumatic pressure or a vacuum to the cartridge when mounted on the seating area;
a thermal module configured to control temperature in a predetermined portion of the cartridge when mounted on the seating area;
an optic module positioned to irradiate light onto the cartridge when mounted on the seating area, and detect light generated or reflected from a sample inside the cartridge when mounted on the seating area;
a fluid sensing module that determines whether a fluid in a predetermined portion of a cartridge mounted on the seating area is in a gaseous state or a liquid state;
a scanning module that moves the optic module and the fluid sensing module relative to the seating area; and
a control module that controls operations of the pneumatic module, the thermal module, the optic module, the fluid sensing module, and the scanning module, and processes and analyzes data received therefrom.

2. The nucleic acid analysis system of claim 1, wherein the fluid sensing module comprises:
a light source positioned to emit light toward a cartridge when mounted on the seating area; and
a photodetector positioned to detect light generated or reflected from a sample within the cartridge.

3. The nucleic acid analysis system of claim 2, wherein the light source comprises a light emitting diode or a laser diode, and the photodetector comprises a photodiode, a photomultiplier tube, a phototransistor, a charge-coupled device (CCD) image sensor, or a complementary metal-oxide-semiconductor (CMOS) image sensor.

4. The nucleic acid analysis system of claim 2, wherein the fluid sensing module further comprises a reflector positioned to reflect light that passes through the cartridge to the photodetector.

5. The nucleic acid analysis system of claim 4, wherein the light source and the photodetector are disposed opposite the reflector relative to a cartridge when mounted on the seating area.

6. The nucleic acid analysis system of claim 2, wherein the control module is configured to determine a state of a fluid in a predetermined portion of a cartridge when mounted on the seating area by comparing a reference value to a signal from the fluid sensing module.

7. The nucleic acid analysis system of claim 6, wherein the signal from the fluid sensing module is an average value of a plurality of signals from the fluid sensing module.

8. The nucleic acid analysis system of claim 6, wherein the control module is configured to determine a state of the fluid by comparing a reference value with the difference between an average value of a plurality of signals from the fluid sensing module during a first time period and an average value of a plurality of signals from the fluid sensing module during a second time period earlier than the first time period.

9. The nucleic acid analysis system of claim 6, wherein the control module is configured to determine a state of the fluid by comparing a reference value with a temporal differential value calculated using a signal measured by the fluid sensing module at a first time point, and a signal measured by the fluid sensing module a predetermined number of times prior to the first time point.

10. The nucleic acid analysis system of claim 6, wherein the control module is configured to determine a state of the fluid by comparing a reference value with a temporal differential value calculated using an average value of a plurality of data obtained from reflected light measured by the fluid sensing module and an average value of a plurality of data obtained from reflected light measured a predetermined number of times previously by the fluid sensing module.

11. The nucleic acid analysis system of claim 6, wherein the control module is configured to determine that a state of the fluid has changed after a signal indicating that a state of the fluid has changed occurs consecutively at least a predetermined number of measurement times.

12. The nucleic acid analysis system of claim 1, wherein the pneumatic module comprises:
a pneumatic pump that generates a pneumatic pressure;
a vacuum pump that generates a vacuum;
a chamber connected to the pneumatic pump and vacuum pump;
a regulator that regulates the pneumatic pressure of the chamber; and
a plurality of pneumatic ports that are configured to inject a pneumatic pressure and a vacuum into a cartridge mounted on the seating area.

13. The nucleic acid analysis system of claim 1, wherein the thermal module comprises:
a heating unit that is configured to raise a temperature of a predetermined portion of a cartridge when mounted on the seating area; and
a cooling unit that is configured to reduce a temperature of a predetermined portion of a cartridge when mounted on the seating area.

14. The nucleic acid analysis system of claim 1, wherein the optic module detects fluorescence.

15. The nucleic acid analysis system of claim 14, wherein the optic module comprises:
a light source positioned to irradiate excitation light onto a cartridge when mounted on the seating area; and
a photodetector positioned to detect fluorescence generated from a fluorescent dye in a sample within a fluid in a cartridge when mounted on the seating area.

16. The nucleic acid analysis system of claim 1, wherein the optic module and the fluid sensing module are coupled to the scanning module.

17. The nucleic acid analysis system of claim 1, wherein the control module comprises:
a microprocessor and a non-transitory storage medium storing an algorithm for controlling the pneumatic module, the thermal module, the optic module, the fluid sensing module, and the scanning module and analyze data.

18. A method for nucleic acid analysis comprising:
mounting a cartridge comprising a sample on the seating area of the system of claim 1, wherein the sample contains a nucleic acid, and amplifying and detecting a nucleic acid while controlling a temperature of the sample.

19. The nucleic acid analysis method of claim 18, wherein the sample comprises any one or a combination of: a swab, a culture medium, saliva, sputum, a cellular tissue, urine, stool, blood, pus, or cerebrospinal fluid.

20. The nucleic acid analysis method of claim 19, wherein the temperature of the sample comprises:
maintaining a constant temperature for a predetermined time;
changing a temperature within a predetermined range for a predetermined time;
repeatedly maintaining a plurality of different temperatures for respective predetermined times;
or combination thereof.
